(19) United States
(12) Patent Application Publication
(10) Pub. No.: US 2024/0217901 A1
(43) Pub. Date: Jul. 4, 2024

(54) PROCESS AND APPARATUS FOR PRODUCING ETHYLENE FROM ALCOHOL

(71) Applicant: UOP LLC, Des Plaines, IL (US)

(72) Inventors: Saikrishna Laxmirajam Gosangari, Gurugram (IN); Ashish Mathur, Gurgaon (IN); Charles Luebke, Mount Prospect, IL (US); Gautam Pandey, Gurugram (IN); Chintan Surendra Shah, Ahmedabad (IN)

(21) Appl. No.: 18/393,482

(22) Filed: Dec. 21, 2023

Related U.S. Application Data
(60) Provisional application No. 63/477,387, filed on Dec. 28, 2022.

(51) Int. Cl. C07C 5/333; B01J 19/00

(52) U.S. Cl. CPC C07C 5/333; B01J 19/0053

(57) ABSTRACT

A new process and apparatus for ethanol dehydration combines a fired heater with a dehydration reactor in a single vessel. The reactor provides catalyst tubes in a firebox. The catalyst may be added to and withdrawn from the catalyst tubes. The apparatus reduces equipment account substantially while holding the reaction to isothermal conditions.

### FIELD

The field is the conversion of alcohols to olefins. The field may particularly relate to the dehydration of ethanol to produce ethylene and the subsequent conversion of the ethylene to long chain olefins and the hydrogenation of the long chain olefins to produce paraffins.

### BACKGROUND

Oil and gas refiners worldwide are exploring methodologies and routes to reduce the carbon footprint in more sustainable processes. An ethanol to jet fuel process is one of the routes that holds promise to minimize or eliminate net carbon combustion. The end product of this process is jet and diesel fuel produced out of bioethanol. The jet fuel is a sustainable aviation fuel intended to replace jet fuel produced out of conventional sources such as crude oil.

Three main steps are followed in the process to convert ethanol to jet fuel. The first is to dehydrate ethanol to produce ethylene. Next the ethylene is converted to long chain olefins and then the long chain olefins are hydrogenated to generate paraffins.

The ethanol dehydration process involves dehydration of ethanol molecules to generate ethylene and water. The process of converting ethanol to ethylene is endothermic in nature and the heat of endothermicity is typically provided by fired heaters that are adiabatic in nature. Adiabatic reactor systems may have drawbacks such as selectivity to undesired products, potential underutilization of catalyst, higher utility consumption and larger plot space. An improved reactor would be desirable for dehydrating of ethanol to ethylene.

### SUMMARY OF THE INVENTION

We have discovered a new process and apparatus for ethanol dehydration that combines a fired heater with a dehydration reactor in a single vessel. The reactor provides catalyst tubes in a firebox. The catalyst may be added to and withdrawn from the catalyst tubes.

### BRIEF DESCRIPTION OF THE DRAWINGS

FIG. 1 is a schematic process flow diagram of the present disclosure.

FIG. 2 is a schematic illustration of the reactor of the present disclosure.

FIG. 3 is a partial view of FIG. 2 depicting an additional embodiment of the present disclosure.

### DEFINITIONS

The term “communication” means that material flow is operatively permitted between enumerated components.

The term “downstream communication” means that at least a portion of material flowing to the subject in downstream communication may operatively flow from the object with which it communicates.

The term “upstream communication” means that at least a portion of the material flowing from the subject in upstream communication may operatively flow to the object with which it communicates.

The term “direct communication” means that flow from the upstream component enters the downstream component without passing through a fractionation or conversion unit to undergo a compositional change due to physical fractionation or chemical conversion.

The term “indirect communication” means that flow from the upstream component enters the downstream component after passing through a fractionation or conversion unit to undergo a compositional change due to physical fractionation or chemical conversion.

The term “bypass” means that the object is out of downstream communication with a bypassing subject at least to the extent of bypassing.

The term “column” means a distillation column or columns for separating one or more components of different volatilities. Unless otherwise indicated, each column includes a condenser on an overhead of the column to condense and reflux a portion of an overhead stream back to the top of the column and a reboiler at a bottom of the column to vaporize and send a portion of a bottoms stream back to the bottom of the column. Feeds to the columns may be preheated. The top pressure is the pressure of the overhead vapor at the vapor outlet of the column. The bottom temperature is the liquid bottom outlet temperature. Overhead lines and bottoms lines refer to the net lines from the column downstream of any reflux or reboil to the column. Stripper columns may omit a reboiler at a bottom of the column and instead provide heating requirements and separation impetus from a fluidized inert media such as steam. Stripping columns typically feed a top tray and take main product from the bottom.

As used herein, the term “a component-rich stream” means that the rich stream coming out of a vessel has a greater concentration of the component than the feed to the vessel.

As used herein, the term “a component-lean stream” means that the lean stream coming out of a vessel has a smaller concentration of the component than the feed to the vessel.

As used herein, the term “separator” means a vessel which has an inlet and at least an overhead vapor outlet and a bottoms liquid outlet and may also have an aqueous stream outlet from a boot. A flash drum is a type of separator which may be in downstream communication with a separator that may be operated at higher pressure.

As used herein, the term “predominant” or “predominate” means greater than 50%, suitably greater than 75% and preferably greater than 90%.

As used herein, the term “Cx” are to be understood to refer to molecules having the number of carbon atoms represented by the subscript “x”. Similarly, the term “Cx-” refers to molecules that contain less than or equal to x and preferably x and less carbon atoms. The term “Cx+” refers to molecules with more than or equal to x and preferably x and more carbon atoms.

As used herein, the term “carbon number” refers to the number of carbon atoms per hydrocarbon molecule and typically a paraffin molecule.

### DETAILED DESCRIPTION

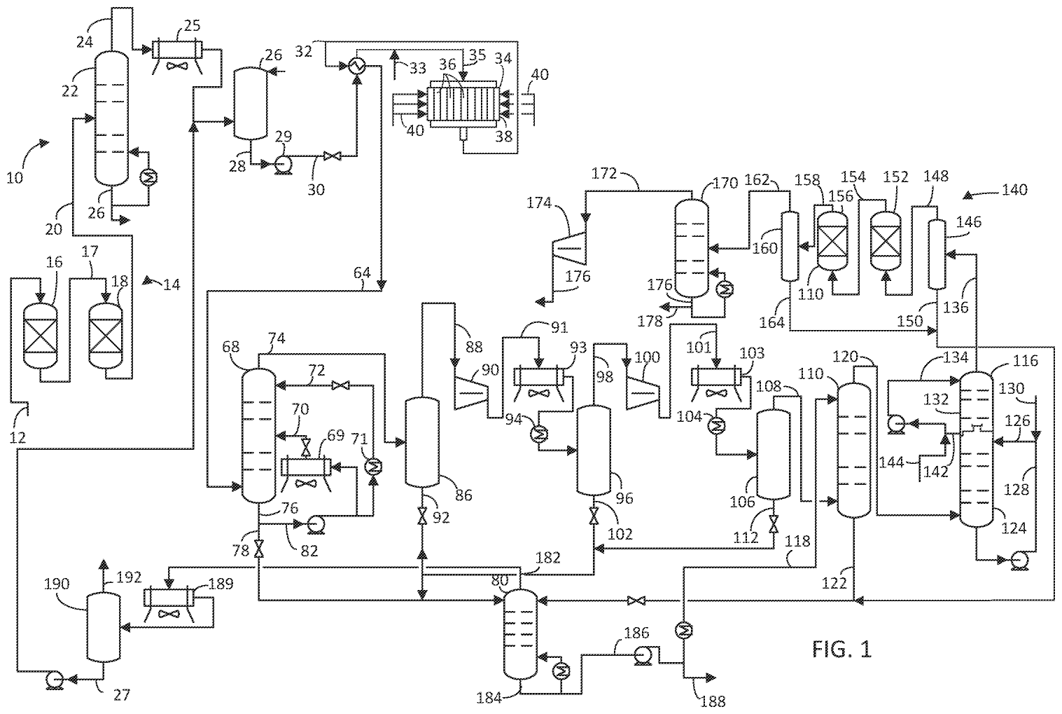

In FIG. 1, in accordance with an exemplary embodiment, a process and apparatus 10 are shown for dehydrating an oxygenate feedstock. The oxygenate feedstock may comprise alcohol and preferably comprises ethanol. The feedstock may comprise a predominance of ethanol and may be aqueous. Preferably, the oxygenate feedstock is a biorenewable feedstock.

A feed line 12 transports an oxygenate stream of oxygenate feedstock to a feed pretreatment section 14. The feed pretreatment section 14 comprises a vessel 16 comprising a bed of cationic exchange resin adsorbent for removing metal contaminants, such as sodium, zinc, phosphates, copper, and calcium from the oxygenate stream in the feed line 12. The adsorbent may be an Amberlyst 15 available from DuPont. The feed pretreatment section 14 may comprise an additional vessel 18 with a bed of the same adsorbent for further removing metals from the oxygenate stream. The vessels 16, 18 may be in series or in a lead-lag type of arrangement to allow for regeneration of spent adsorbent. Line 17 transports a partially pretreated oxygenate stream from an outlet of vessel 16 to the inlet of vessel 18. A pretreated oxygenate stream exits the feed pretreatment section 14 in line 20 from an outlet of the additional vessel 18 and is fed to a purification column 22. The feed pretreatment section 14 may be operated at a temperature of about 32° ° C. (90° F.) to about 104° C. (220° F.) and a pressure of about atm to about 696 kPa (gauge) (100 psig).

In the purification column 22, the pretreated oxygenate stream is fractionated to separate ethanol from heavier oxygenates also known as fusel oil such as cyclohexanol, cyclopentanol, and heavier acids. The purification column 22 is operated to minimize ethanol to no more than 1% of feed in bottom stream in line 26. A heavy oxygenate stream in a bottoms line 26 is taken from a bottom of the purification column 22 to heavy oxygenate treatment. The purification column 22 may be reboiled by heat exchange with a suitable hot stream such as steam to provide the necessary heat for the distillation. The purification column 22 provides an overhead gaseous stream of purified ethanol in an overhead line 24 which may be cooled in an air cooler 25 and fed to a feed surge drum 26 along with a recycle ethanol stream in line 27. The purification column 22 may be operated with a bottoms temperature between about 82° C. (180° F.) and about 121° ° C. (250° F.) and an overhead pressure of about 35 kPa (gauge) (5 psig) to about 140 kPa (gauge) (20 psig).

Ethanol in the feed surge drum 26 may be blanketed with nitrogen. A charge pump 29 pumps an ethanol charge stream in line 28. The ethanol charge stream in line 28 is heat exchanged with a dehydrated exchange stream in line 32, mixed with steam in line 33 and fed to a dehydration reactor 34 in a charge line 35. In the dehydration reactor 34, the ethanol charge stream in line 28 is charged to a plurality of catalyst tubes 36 comprising dehydration catalyst. The catalyst tubes 36 are located in a fire box 38 in which a fuel stream from fuel lines 40 combust to provide heat to supply enthalpy for the endothermic dehydration reaction. In the dehydration reactor 34, ethanol feed is converted to ethylene and water over a dehydration catalyst at about 400° ° C. to about 550° C. and at a pressure of about 455 kPa (gauge) 65 psig to about 630 kPa (gauge) (90 psig) in the catalyst tubes 36. A dehydrated stream is discharged from the dehydration reactor 34 in an effluent line 32. The dehydration catalyst is an alumina-based catalyst.

The dehydrated stream in line 32 is heat exchanged with the charge stream in line 30 to provide a cooled dehydrated stream in line 64. The cooled dehydrated stream in line 64 is fed to a quench tower 68 in which the cooled dehydrated stream is quenched by direct contact with water from a first cooled water stream in line 70 and a second cooled water stream in line 72. A quenched ethylene stream exits in a quench overhead line 74 and a bottoms water stream exits the tower bottoms in line 76. The bottoms water stream is split between a drain stream in line 78 which may be transported to a waste-water stripper column 80 through a valve thereon and a quench recycle stream in line 82. A first portion of the quench recycle stream is air cooled in a product condenser 69 and recycled as the first, lower cooled water stream in line 70 through a valve thereon, and a second portion of the quench recycle stream is heat exchanged in a trim condenser 71 and recycled to the quench tower 68 as the second, higher cooled water stream in line 72. The quench tower 68 may be operated with a bottoms temperature of about 37° C. (100° F.) to about 104° C. (220° F.) and a pressure of about 280 kPa (gauge) (40 psig) to about 490 kPa (gauge) (70 psig) in the overhead.

The quenched ethylene stream in line 74 is fed to a first stage suction drum 86. In the first stage suction drum ethylene exits in the overhead line 88 to a first stage compressor 90 while residual water exits the bottom of the drum in line 92 through a control valve thereon and is transported to the waste-water stripper column 80 perhaps via line 78. The first stage compressor 90 compresses the ethylene stream to a first pressure of about 350 kPa (gauge) (50 psig) to about 1225 kPa (gauge) (175 psig) and the discharge in line 91 is cooled in a first stage discharge cooler 93 and a first stage trim cooler 94.

The cooled, compressed ethylene stream from the first stage trim cooler 94 is fed to a first stage discharge drum 96. From the first stage discharge drum 96 ethylene exits in an overhead line 98 to a second stage compressor 100 while residual water exits a bottom of the drum in line 102 through a control valve thereon and is transported to the waste-water stripper column 80 perhaps via lines 92 and 78. The second stage compressor compresses the ethylene stream to a second pressure of about 455 kPa (gauge) (165 psig) to about 3220 kPa (gauge) (460 psig) and the discharge in line 101 is cooled in a second stage discharge cooler 103 and a second stage trim cooler 104.

The twice cooled, compressed ethylene stream from the second stage trim cooler 104 is fed to a second stage discharge drum 106. From the second stage discharge drum 106 ethylene exits in an overhead line 108 and is transported to a water wash tower 110 while a residual water stream exits the bottom of the drum in line 112 through a control valve thereon and is transported to the waste-water stripper column 80 perhaps via lines 102, 92 and 78.

In the water wash tower 110, the twice cooled, compressed ethylene stream is counter-currently washed with cooled, treated water in line 118 from the waste-water stripper column 80 to absorb additional oxygenates to produce a washed ethylene stream exiting in an overhead line 120 and a wash water stream in a bottoms line 122. The washed ethylene stream in the overhead line 120 is transported to a caustic scrubber column 116. The wash water stream in line 122 is transported back to the waste-water stripper column 80 through a valve thereon. The water wash tower 110 may be operated with a bottoms temperature of about 16° C. (60° F.) to about 82° ° C. (150° F.) and a pressure of about 2800 kPa (gauge) (400 psig) to about 3500 kPa (gauge) (500 psig) in the overhead.

The caustic scrubber column 116 has a lower caustic wash section 124 and an upper water wash section 132. In the lower caustic wash section 124 the washed ethylene stream in line 120 is scrubbed with an aqueous caustic stream from line 126 to absorb acid gases such as carbon dioxide from the washed ethylene stream. Spent caustic is pumped around from the bottom of the lower section in line 128 and replenished with fresh caustic in line 130 to provide the aqueous caustic stream 126. A scrubbed vaporous ethylene stream depleted of acid gases ascends from the caustic wash section 124 to the upper water wash section 132 through a vapor inlet. In the water wash section 132, the scrubbed ethylene stream is contacted with a wash water stream from line 134. A washed, scrubbed vaporous ethylene stream exits the overhead of the water wash section 132 in line 136 and is fed to the product drier section 140. A spent water stream is taken from the bottom of the water wash section 132 from a liquid sump in line 142 and replenished with a fresh water stream from line 144 to provide the wash water stream in line 134 and pumped to the top of the water wash section 132 to be contacted with the scrubbed vaporous ethylene stream. The caustic scrubber column may be operated with a bottoms temperature of about 38° C. (100° F.) to about 43° C. (110° F.) and a pressure of about 2800 kPa (gauge) (400 psig) to about 2975 kPa (gauge) (425 psig) in the overhead.

In the product drier section 140, the washed, scrubbed ethylene stream in line 136 is fed to a first drier inlet knock-out drum 146 to remove residual water and provide a drier inlet stream in line 148 and a knock-out water stream in the bottoms line 150 which is fed to the waste-water stripper column 80 perhaps via line 122. The drier inlet stream is fed to a first product drier 152 in line 148. The first product drier 152 comprises an adsorbent for adsorbing the water from ethylene in the drier inlet stream in line 148 to provide a dried ethylene stream. The adsorbent may be a molecular sieve material with pore diameters of 2-4 A. The first product drier 152 may operate in upflow mode. The product drier section 140 may include a second product drier 156 that operates as the first product drier 142. The two product driers may be operated in series but are preferably arranged in a lead-lag operation to facilitate regeneration during continuous operation. The second product drier 156 comprises an adsorbent for adsorbing the water from ethylene like in the first product drier 152. A dried ethylene stream exits the product drier section 140 in a dried ethylene stream in line 158. The product drier section 140 may be operated at a temperature of about 32° ° C. (90° F.) to about 49° C. (120° F.) and a pressure of about 2.8 MPa (gauge) (400 psig) to about 3.1 MPa (gauge) 450 psig).

The dried ethylene stream in line 158 is fed to a drier outlet knock-out drum 160 to remove residual water and provide a drier outlet stream in line 162 and a second knock-out water stream in a bottoms line 164 which is fed to the waste-water stripper column 80 perhaps via lines 150 and 122.

The drier outlet stream in line 162 may be fed to a heavy oxygenates removal column 170 to separate an overhead stream comprising predominantly ethylene but perhaps higher olefins from heavy ketones and diethyl ether. The olefins are produced in an overhead line 172 and fed to a third stage compressor 174 and a bottoms heavy oxygenate stream is produced in a bottoms line 176. A heavy oxygenate purge stream may be taken in line 178 to heavy oxygenate treatment while a reboil portion is reboiled and fed back to the column 170. A compressed ethylene stream at a pressure of about 2800 kPa (gauge) (400 psig) to about 7000 kPa (gauge) (1000 psig) in a compressor discharge line 176 may be provided to a dimerization section. The heavy oxygenate removal column 170 may be operated with a bottoms temperature of about −29° ° C. (−20° F.) to about 121° C. (250° F.) and a pressure of about 2.4 MPa (gauge) (350 psig) to about 3.1 MPa (gauge) (450 psig) in the overhead.

Water streams comprising oxygenates and volatiles in lines 92, 102, 112, 122, 150, 164 may be fed to the waste-water stripper column 80 in which volatiles and oxygenates are boiled off to provide an overhead volatile stream in line 182 and a stripped water stream in line 184. A portion of the stripped water stream can be reboiled and fed back to the column to provide necessary heat. A treated water stream in line 186 may be pumped to water outlets which includes other water outlets in line 188 and the cooled, treated water stream in line 118 to the water wash tower 110. The waste-water stripper column 80 may be operated with a bottoms temperature of about 93° C. (200° F.) to about 121° C. (250° F.) and a pressure of about 35 kPa (gauge) (5 psig) to about 138 kPa (gauge) (20 psig) in the overhead.

The overhead volatile stream in line 182 may be cooled in an air cooler 189 and fed to an off-gas knock out drum 190. An overhead stream from the knockout drum 190 in line 192 may be sent to flare while an ethanol recycle stream is pumped to the feed surge drum 26 in line 27 perhaps via line 24.

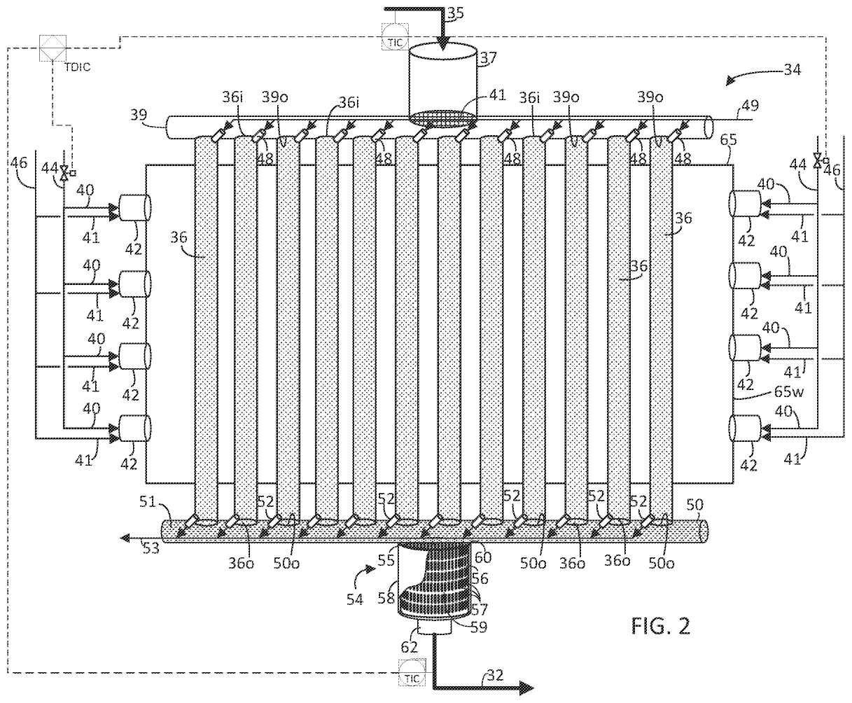

The dehydration reactor 34 is shown in greater detail in FIG. 2. The ethanol charge stream in line 35 charges heated ethanol to a reactor inlet 37. A temperature indicator controller (TIC) may be located on the line 35 for measuring the temperature of the ethanol charge stream. The distribution inlet 37 charges ethanol to a distribution header 39 through a screened port 41. The distribution header 39 may be a pipe grid or it may comprise a cylindrical or rectangular box with a tube sheet mated to the catalyst tubes 36. Nevertheless, the distribution header has distribution openings 390 that are contiguous with catalyst tube inlets 36i to a respective catalyst tube 36. The distribution openings 390 may be mated to the distribution header 39 by a mated flanged connection (not shown). Moreover, a mechanical bellow may be utilized at the flanged connection for case of thermal expansion and assembly of the distribution header and the catalyst tubes. The distribution header may be in upstream communication with catalyst tube inlets 36i to the catalyst tubes 36 for distributing ethanol to the catalyst tubes 36. The distribution header 39 may be at the top of the dehydration reactor 34. The distribution openings 390 may be on the bottom of the distribution header 39, and the catalyst tube inlets 36i may be at the top of the catalyst tubes 36. The distribution header 39 distributes charged ethanol from the distribution openings 390 through the catalyst tube inlets 36i into the catalyst tubes 36.

The catalyst tubes 36 are filled with dehydration catalyst for converting the ethanol to ethylene. A collection header 50 collects ethylene from the catalyst tubes 36. The collection header 50 may be a pipe grid or it may comprise a cylindrical or rectangular box with a tube sheet mated to the catalyst tubes 36. Nevertheless, the collection header 50 has collection openings 500 that are contiguous with catalyst tube outlets 360 from a respective catalyst tube 36. The openings 500 may have a single screen or a series of screens to hold catalyst in the catalyst tube 36 itself. The collection header 50 may be in downstream communication with catalyst tube outlets 360 for collecting ethylene product from the catalyst tubes 36. The collection header 50 may be at the bottom of the dehydration reactor 34. The collection openings 500 may be on the top of the collection header 50, and the catalyst tube outlets 360 may be at the bottom of the catalyst tubes 36. The collection header 50 collects product ethanol from the catalyst tube outlets 360 of the catalyst tubes 36 through the collection openings 500.

The catalyst tubes 36 extend through a firebox 65. The firebox 65 may be cylindrical, or it may be rectangular. The catalyst tubes 36 may be suspended along the vertical wall(s) 65w of the firebox 65. Alternatively, the catalyst tubes 36 may be placed toward the center, so the catalyst tubes 36 are exposed to burners 42 on either side or surrounding the catalyst tubes in the firebox 65. The vertical wall 65w may be cylindrical or planar. A top wall and a bottom wall enclose the firebox 65 which may provide a tube sheet that allows the catalyst tubes 36 to extend therethrough. The distribution header 39 and the collection header 50 may be outside of the firebox 65. The distribution header 39, the distribution openings 390 and the catalyst tube inlets 36i may be above the firebox 65. The collection header 50, the collection openings 500 and the catalyst tube outlets 360 may be below the firebox 65. The firebox encloses a predominance of the length of each of the catalyst tubes 36.

Burners 42 may be disposed in the vertical wall(s) 65w of the firebox 65. The vertical wall can be either a side wall or an end wall of the firebox 65. The burners 42 may also be disposed either on the top wall or bottom wall or any combination thereof in the firebox 65. A fuel manifold 44 delivers a hydrocarbon fuel at a flow rate regulated by a control valve thereon to each burner 42 through the fuel lines 40. An air manifold 46 delivers air to each burner 42 through air lines 41 as well. The fuel ignites with the air and combusts in the firebox 65 to provide enthalpy to the endothermic dehydration reaction occurring in the catalyst tubes 36.

Each catalyst tube 36 has a catalyst inlet 48 near the inlet 36i of the catalyst tube and a catalyst outlet 52 near the outlet 360 of the catalyst tube. The catalyst inlet 48 may be at a top of the catalyst tube 36 and the catalyst outlet 52 may be at the bottom of the catalyst tube. Fresh catalyst is supplied by a fresh catalyst manifold 49 through the catalyst inlet 48 to the catalyst tubes 36, and spent catalyst is withdrawn from the catalyst outlet 52 from the catalyst tubes to a spent catalyst manifold 53. The catalyst inlets 48 and the catalyst outlets 52 may be outside of the firebox 65 to facilitate on-stream provision and withdrawal of catalyst to and from the catalyst tubes 36, respectively. The tube inlet 36i and the tube outlet 360 may be equipped with screens to prevent catalyst from passing out of the catalyst tubes except through the catalyst inlet 48 and the catalyst outlet 52, respectively. The screens may have openings smaller that the smallest dimension of the catalyst to prevent passage therethrough. The collection header 50 may be loaded with inert balls or other inert particulate 51 to support screens in the catalyst outlets 360. The inert balls may be made of ceramic.

A collection basket 54 may be in downstream communication with the collection header 50. An inlet 55 to the collection basket 54 may be equipped with a screen to prevent entry by inert particulates from the collection header 50. The collection basket 54 may be cylindrical. The collection basket 54 may comprise an inner perforated wall 56 with perforations 57 therein. The inner perforated wall 56 defines a collection chamber 57 therewithin. An outer imperforate wall 58 outside of the inner perforate wall 56 defines an annulus 60 therebetween. The outer imperforate wall 58 is only partially shown to reveal the inner perforate wall 56. The collection basket 54 may be secured to the collection header by a flanged connection in which a flange of the collection basket 54 is sandwiched between a flange depending from the collection header 50 and a flange of the outer imperforate wall 58. In FIG. 2, a closed side of the basket 54 is in downstream communication with an open side of the basket. In an alternative embodiment, the collection basket 54 may have an open side downstream of its closed side.

Product ethylene flows from said collection header 50 to the collection chamber 59 defined by said inner perforate wall through the perforations 57 in the inner perforate wall 56, into the annulus 60 and to a discharge nozzle 62 in downstream communication with the annulus. The annulus 60 is preferably in downstream communication with said collection chamber 59, but the annulus may be in upstream communication with the collection chamber 59 if the basket extends toward the collection header 50. In the embodiment of FIG. 2, the product ethylene flows from the collection chamber 59 into the annulus 60, but the obverse is contemplated. The effluent line 32 evacuates product ethylene from the reactor 34. A temperature indicator controller (TIC) may be located on the line 32 for measuring the temperature of the ethylene effluent stream.

The TIC on the charge line 35 may measure the temperature of the charge stream entering the dehydration reactor 34, and the TIC on the effluent line 32 may measure the temperature of the dehydrated stream. The respective temperatures may be sent as a signal to a temperature differential indicator controller (TDIC) which calculates the temperature differential and compares it to a set point. The TDIC may signal a control valve on the fuel manifold 44 to open more to increase the temperature differential if below or close to the set point or close more to decrease the temperature differential if the temperature differential is above or close to the set point.

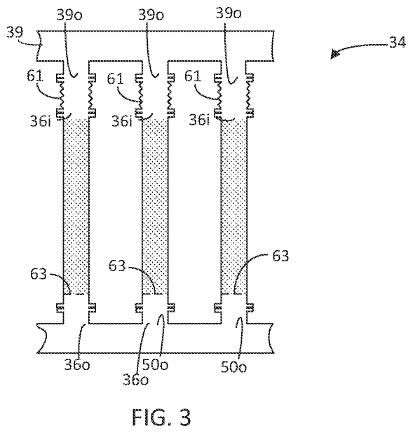

FIG. 3 is a partial view of FIG. 2 depicting an additional embodiment of the catalyst tubes 36 in the reactor 34. The distribution header 39 has flanged connections to the catalyst tubes between each of the distribution openings 390 and the respective catalyst tube inlet 36i. The distribution header 39 can be configured to have each of the distribution openings 390 to be flanged connected with a mechanical bellow 61 in connection with the respective catalyst tube inlet 36i. The mechanical bellow 61 facilitates differential thermal expansion and installation. The distribution header 39 can be swinged open to allow for catalyst loading and unloading from the catalyst tube inlet 36i. The collection header 50 has a flanged connection to the catalyst tubes 36 between each of the collection openings 500 and the respective catalyst tube outlet 360. The catalyst tubes and/or collection openings 500 may have a single screen 63 or a series of screens to support catalyst in the catalyst tube 36 itself. This configuration may reduce the complexity of or the need for the collection basket 54.

The process and apparatus disclosed provide a dehydration reactor 34 that combines a fired heater and a reactor within a single piece of equipment, which reduces equipment count and plot space. The single dehydration reactor 34 may provide an isothermal reactor which can increase selectivity to desired ethanol through efficient catalyst utilization and reduced utility requirements.

### EXAMPLE

We developed a kinetic model and compared the reactor of the present disclosure to the adiabatic reactor for ethanol dehydration. The model assumed a feed rate of 300 M gallons per year, steam rate of about 300,000 lb/hr and ethanol conversion of about 98% per pass. The Table below shows the improvement provide by the isothermal reactor of the present disclosure.

TABLE

| | Adiabatic Reactors | Isothermal |
|---|---|---|
| Reactor Type | in Series | Reactor |
| Catalyst Volume | Base | 50% of Base |
| Number of Fired Heaters and | 6 | 1 |
| Reactors | | |
| Average Inlet to Outlet Reactor | 200 | 0 |
| Temperature Delta, ° F. | | |

### SPECIFIC EMBODIMENTS

While the following is described in conjunction with specific embodiments, it will be understood that this description is intended to illustrate and not limit the scope of the preceding description and the appended claims.

A first embodiment of the disclosure is a process for producing ethylene comprising charging ethanol to a plurality of catalyst tubes comprising dehydration catalyst in a firebox to convert ethanol to ethylene; and combusting a fuel in the firebox around the tubes. An embodiment of the disclosure is one, any or all of prior embodiments in this paragraph up through the first embodiment in this paragraph further comprising charging the ethanol to a distribution header that distributes the ethanol to the tubes. An embodiment of the disclosure is one, any or all of prior embodiments in this paragraph up through the first embodiment in this paragraph further comprising collecting ethylene from the catalyst tubes in a collection header. An embodiment of the disclosure is one, any or all of prior embodiments in this paragraph up through the first embodiment in this paragraph further comprising supplying catalyst through a catalyst inlet to the catalyst tubes. An embodiment of the disclosure is one, any or all of prior embodiments in this paragraph up through the first embodiment in this paragraph wherein the catalyst inlet is outside of the firebox. An embodiment of the disclosure is one, any or all of prior embodiments in this paragraph up through the first embodiment in this paragraph further comprising withdrawing catalyst from the catalyst tubes through a catalyst outlet. An embodiment of the disclosure is one, any or all of prior embodiments in this paragraph up through the first embodiment in this paragraph wherein the catalyst outlet is outside of the firebox. An embodiment of the disclosure is one, any or all of prior embodiments in this paragraph up through the first embodiment in this paragraph further comprising inert particulates in the collection header. An embodiment of the disclosure is one, any or all of prior embodiments in this paragraph up through the first embodiment in this paragraph further comprising a collection basket in downstream communication with the collection header, the collection basket having an inner perforated wall and an outer imperforate wall defining an annulus therebetween. An embodiment of the disclosure is one, any or all of prior embodiments in this paragraph up through the first embodiment in this paragraph wherein ethylene flows from the collection header to a collection chamber defined by the inner perforated wall, through perforations in the inner perforated wall, through an annulus to a discharge nozzle.

A second embodiment of the disclosure is an apparatus for producing ethylene comprising a firebox comprising burners in the side of the firebox; catalyst tubes in the firebox; a distribution header in communication with an inlet to the catalyst tubes for distributing ethanol to the catalyst tubes; a catalyst inlet to the catalyst tubes; a collection header in communication with an outlet from the catalyst tubes for collecting ethylene from the catalyst tubes; and a catalyst outlet from the catalyst tubes. An embodiment of the disclosure is one, any or all of prior embodiments in this paragraph up through the second embodiment in this paragraph wherein the catalyst inlet is outside of the firebox. An embodiment of the disclosure is one, any or all of prior embodiments in this paragraph up through the second embodiment in this paragraph wherein the catalyst outlet is outside of the firebox. An embodiment of the disclosure is one, any or all of prior embodiments in this paragraph up through the second embodiment in this paragraph wherein the distribution header is outside of the firebox. An embodiment of the disclosure is one, any or all of prior embodiments in this paragraph up through the second embodiment in this paragraph wherein the collection header is outside of the firebox. An embodiment of the disclosure is one, any or all of prior embodiments in this paragraph up through the second embodiment in this paragraph further comprising a collection basket in downstream communication with the collection header, the collection basket having an inner perforated wall and an outer imperforate wall defining an annulus therebetween.

A third embodiment of the disclosure is a process for producing ethylene comprising charging ethanol to a distribution header that distributes the ethanol to a plurality of catalyst tubes comprising dehydration catalyst in a firebox to convert ethanol to ethylene; combusting a fuel in the firebox around the tubes; collecting ethylene from the catalyst tubes in a collection header. An embodiment of the disclosure is one, any or all of prior embodiments in this paragraph up through the third embodiment in this paragraph further comprising supplying catalyst through a catalyst inlet to the catalyst tubes at a top of the catalyst tubes. An embodiment of the disclosure is one, any or all of prior embodiments in this paragraph up through the third embodiment in this paragraph further comprising withdrawing catalyst from the catalyst tubes through a catalyst outlet at a bottom of the catalyst tubes. An embodiment of the disclosure is one, any or all of prior embodiments in this paragraph up through the third embodiment in this paragraph wherein the catalyst inlet and the catalyst outlet are outside of the firebox.

Without further elaboration, it is believed that using the preceding description that one skilled in the art can utilize the present invention to its fullest extent and easily ascertain the essential characteristics of this invention, without departing from the spirit and scope thereof, to make various changes and modifications of the invention and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

1. A process for producing ethylene comprising:
charging ethanol to a plurality of catalyst tubes comprising dehydration catalyst in a firebox to convert ethanol to ethylene; and
combusting a fuel in the firebox around the tubes.

2. The process of claim 1 further comprising charging the ethanol to a distribution header that distributes the ethanol to the tubes.

3. The process of claim 2 further comprising collecting ethylene from the catalyst tubes in a collection header.

4. The process of claim 2 further comprising supplying catalyst through a catalyst inlet to the catalyst tubes.

5. The process of claim 4 wherein said catalyst inlet is outside of the firebox.

6. The process of claim 4 further comprising withdrawing catalyst from the catalyst tubes through a catalyst outlet.

7. The process of claim 6 wherein said catalyst outlet is outside of the firebox.

8. The process of claim 3 wherein the tubes comprise a bellows at a catalyst inlet.

9. The process of claim 8 further comprising a collection basket in downstream communication with said collection header, said collection basket having an inner perforated wall and an outer imperforate wall defining an annulus therebetween.

10. The process of claim 9 wherein ethylene flows from said collection header to a collection chamber defined by said inner perforated wall, through perforations in said inner perforated wall, through an annulus to a discharge nozzle.

11. An apparatus for producing ethylene comprising:
a firebox comprising burners in the side of the firebox;
catalyst tubes in the firebox;
a distribution header in communication with an inlet to the catalyst tubes for distributing ethanol to the catalyst tubes;
a catalyst inlet to the catalyst tubes;
a collection header in communication with an outlet from the catalyst tubes for collecting ethylene from the catalyst tubes; and
a catalyst outlet from said catalyst tubes.

12. The apparatus of claim 11 wherein said catalyst inlet is outside of said firebox.

13. The apparatus of claim 11 wherein said catalyst outlet is outside of said firebox.

14. The apparatus of claim 11 wherein said distribution header is outside of said firebox.

15. The apparatus of claim 11 wherein said collection header is outside of said firebox.

16. The apparatus of claim 11 further comprising a collection basket in downstream communication with said collection header, said collection basket having an inner perforated wall and an outer imperforate wall defining an annulus therebetween.

17. A process for producing ethylene comprising:
charging ethanol to a distribution header that distributes the ethanol to a plurality of catalyst tubes comprising dehydration catalyst in a firebox to convert ethanol to ethylene;
combusting a fuel in the firebox around the tubes;
collecting ethylene from the catalyst tubes in a collection header.

18. The process of claim 17 further comprising supplying catalyst through a catalyst inlet to the catalyst tubes at a top of the catalyst tubes.

19. The process of claim 18 further comprising withdrawing catalyst from the catalyst tubes through a catalyst outlet at a bottom of the catalyst tubes.

20. The process of claim 19 wherein said catalyst inlet and said catalyst outlet are outside of the firebox.
